(19) United States
(12) Patent Application Publication
(10) Pub. No.: US 2022/0304348 A1
(43) Pub. Date: Sep. 29, 2022

(54) TRITERPENE GLUCURONIDES AND THEIR USE AS FLAVOR MODIFIERS

(71) Applicant: Firmenich SA, Satigny (CH)

(72) Inventors: Eric FREROT, Satigny (CH); Tim VERNET, Satigny (CH); Isabelle CAYEUX, Satigny (CH); Christine SAINT-LEGER, Satigny (CH); Dattatreya BANAVARA, Plainsboro, NJ (US)

(73) Assignee: Firmenich SA, Satigny (CH)

(21) Appl. No.: 17/640,572

(22) PCT Filed: Dec. 14, 2020
(86) PCT No.: PCT/EP2020/086027

§ 371 Date: Mar. 4, 2022

Related U.S. Application Data
(60) Provisional application No. 62/952,529, filed on Dec. 23, 2019.

(30) Foreign Application Priority Data
Feb. 6, 2020 (EP) 20155959.8

(51) Int. Cl. A23L 27/30; C07J 63/00; A23L 27/00

(52) U.S. Cl. CPC A23L 27/36; C07J 63/008; A23L 27/84; A23L 27/86; A23L 27/88

(57) ABSTRACT

The present disclosure generally provides triterpene glucuronides, and the use of such compounds and related compounds as flavor modifiers. In some aspects, the disclosure provides certain compositions that include such triterpene glucuronides, such as compositions that include such triterpene glucuronides and one or more other sweeteners. In some other aspects, the disclosure provides methods of reducing the caloric content of a sweetened article, such as a sweetened food or beverage product.

### CROSS-REFERENCE TO RELATED APPLICATIONS

The present application claims the benefit of priority of U.S. Provisional Application No. 62/952,529, filed Dec. 23, 2019, and European Patent Application No. 20155959.8, filed Feb. 6, 2020, both of which are hereby incorporated by reference as though set forth herein in their entireties.

### TECHNICAL FIELD

The present disclosure generally provides triterpene glucuronides, such as monoglucuronides and diglucuronides, and the use of such compounds and related compounds as flavor modifiers. In some aspects, the disclosure provides certain compositions that include such triterpene glucuronides, such as compositions that include such triterpene glucuronides and one or more other sweeteners. In some other aspects, the disclosure provides methods of reducing the caloric content of a sweetened article, such as a sweetened food or beverage product.

### DESCRIPTION OF RELATED ART

The taste system provides sensory information about the chemical composition of the external world. Taste transduction is one of the more sophisticated forms of chemically triggered sensation in animals. Signaling of taste is found throughout the animal kingdom, from simple metazoans to the most complex of vertebrates. Mammals are believed to have five basic taste modalities: sweet, bitter, sour, salty, and umami

Sweetness is the taste most commonly perceived when eating foods rich in sugars. Mammals generally perceive sweetness to be a pleasurable sensation, except in excess. Caloric sweeteners, such as sucrose and fructose, are the prototypical examples of sweet substances. Although a variety of no-calorie and low-calorie substitutes exist, these caloric sweeteners are still the predominant means by which comestible products induce the perception of sweetness upon consumption.

Metabolic disorders and related conditions, such as obesity, diabetes, and cardiovascular disease, are major public health concerns throughout the world. And their prevalence is increasing at alarming rates in almost every developed country. Caloric sweeteners are a key contributor to this trend, as they are included in various packaged food and beverage products to make them more palatable to consumers. In many cases, no-calorie or low-calorie substitutes can be used in foods and beverages in place of sucrose or fructose. Even so, these compounds impart sweetness differently from caloric sweeteners, and a number of consumers fail to view them as suitable alternatives. Moreover, such compounds may be difficult to incorporate into certain products. In some instances, they may be used as partial replacements for caloric sweeteners, but their mere presence can cause many consumers to perceive unpleasant off-tastes including, astringency, bitterness, and metallic and licorice tastes. Thus, lower-calorie sweeteners face certain challenges to their adoption.

Sweetness enhancement provides an alternative approach to overcoming some of adoption challenges faced by lower-calorie sweeteners. Such compounds can be used in combination with sucrose or fructose to enhance their sweetness, thereby permitting the use of lower quantities of such caloric sweeteners in various food or beverage products. But, in addition to enhancing the perceived sweetness of the primary sweetener, such compounds nevertheless alter the perceived taste of the sweetener. Thus, many consumers find that it is less pleasurable to consume such sweetness-enhanced products in comparison to unenhanced alternatives having higher calories. Thus, there is a continuing need to discover compounds that enhance the sweetness of caloric sweeteners without altering their perceived taste in a way that detracts from the pleasure that consumers experience in eating or drinking products containing such sweeteners.

### SUMMARY

The present disclosure relates to the discovery that certain compounds exhibit a desirable and surprising sweetness enhancing effect when combined with primary sweeteners at amounts effective to enhance sweetness.

In a first aspect, the disclosure provides flavor-modifying compounds, which are licorice monoglucuronides or diglucuronides. In some embodiments, the licorice monoglucuronides or diglucuronides include licorice monoglucuronides, which are formed by removing one or more glucuronide moieties from licorice polyglucuronides to form licorice monoglucuronides or diglucuronides.

In a second aspect, the disclosure provides flavor-modifying compounds, which are compounds of formula (I):

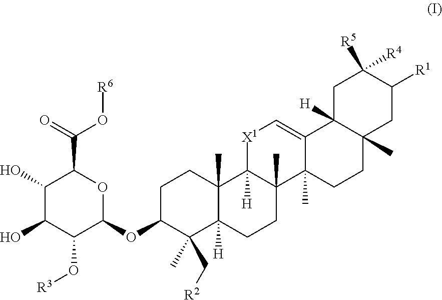

or salts thereof, wherein:

R¹ is a hydrogen atom, —OH, or —O—R^{1A};

R^{1A} is C₁₋₆ alkyl, which is optionally substituted one or more times by substituents selected from the group consisting of —OH and C₁₋₆ alkoxy;

R² is a hydrogen atom, —OH, or —O—R^{2A};

R^{2A} is C₁₋₆ alkyl, which is optionally substituted one or more times by substituents selected from the group consisting of —OH and C₁₋₆ alkoxy; or R^{2A} is —C(O)—(C₁₋₆ alkyl) or a glucosyl moiety;

R³ is a hydrogen atom, a glucuronyl moiety, a C₁₋₆ alkyl glucuronyl ester moiety, or a glucosyl moiety;

one of R⁴ and R⁵ is a methyl group, and the other is —C(O)—O—R^{4A};

R^{4A} is a hydrogen atom, a C₁₋₆ alkyl, a glucuronyl moiety, a C₁₋₆ alkyl glucuronyl ester moiety, or a glucosyl moiety;

R⁶ is a hydrogen atom or C₁₋₆ alkyl; and

X¹ is —CH₂— or >C═O.

In some further embodiments thereof, the flavor-modifying compounds are compounds of formula (Ia):

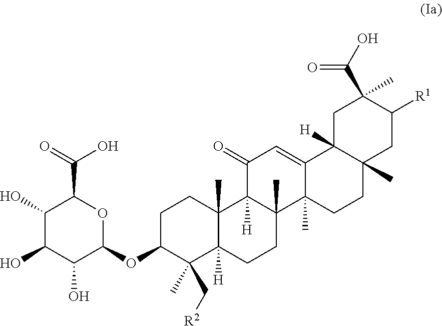

or salts thereof, wherein:

R¹ is a hydrogen atom, —OH, or —O—R^{1A};

R^{1A} is C₁₋₆ alkyl, which is optionally substituted one or more times by substituents selected from the group consisting of —OH and C₁₋₆ alkoxy;

R² is a hydrogen atom, —OH, or —O—R^{2A}; and

R^{2A} is C₁₋₆ alkyl, which is optionally substituted one or more times by substituents selected from the group consisting of —OH and C₁₋₆ alkoxy.

In some embodiments of the foregoing embodiments, at least one of R¹ and R² is not a hydrogen atom. In some such embodiments, one of R¹ and R² is a hydrogen atom, and the other is other is not a hydrogen atom.

In a third aspect, the disclosure provides uses of any flavor-modifying compounds of the first or second aspects.

In a fourth aspect, the disclosure provides uses of any flavor-modifying compounds of the first or second aspects to enhance the sweetness of an ingestible composition. In some embodiments thereof, the ingestible composition comprises a caloric sweetener. In some other embodiments thereof, the ingestible composition comprises a non-caloric sweetener. In some embodiments, the sweetener is a high-intensity sweetener.

In a fifth aspect, the disclosure provides uses of any flavor-modifying compounds of the first or second aspects to reduce the sourness of an ingestible composition.

In a sixth aspect, the disclosure provides uses of any flavor-modifying compounds of the first or second aspects to reduce the bitterness of an ingestible composition.

In a seventh aspect, the disclosure provides uses of any flavor-modifying compounds of the first or second aspects in the manufacture of an ingestible composition to enhance the sweetness of the ingestible composition. In some embodiments thereof, the ingestible composition comprises a caloric sweetener. In some other embodiments thereof, the ingestible composition comprises a non-caloric sweetener. In some embodiments, the sweetener is a high-intensity sweetener. In an eighth aspect, the disclosure provides uses of any flavor-modifying compounds of the first or second aspects in the manufacture of an ingestible composition to reduce the sourness of the ingestible composition.

In a ninth aspect, the disclosure provides uses of any flavor-modifying compounds of the first or second aspects in the manufacture of an ingestible composition to reduce the bitterness of the ingestible composition.

In a tenth aspect, the disclosure provides methods of enhancing the sweetness of an ingestible composition, comprising introducing an amount (such as a sweetness-enhancing effective amount) of any compounds of the first or second aspects to the ingestible composition. In a eleventh aspect, the disclosure provides methods of reducing the sourness of an ingestible composition, comprising introducing an amount (such as a sourness-reducing effective amount) of any compounds of the first or second aspects to the ingestible composition.

In a twelfth aspect, the disclosure provides methods of reducing the bitterness of an ingestible composition, comprising introducing an amount (such as a bitterness-reducing effective amount) of any compounds of the first or second aspects to the ingestible composition.

In a thirteenth aspect, the disclosure provides compositions comprising any compounds of the first or second aspects. In some embodiments, the compounds of the first or second aspects make up at least 20% by weight of the compositions on a dry weight basis (e.g., based on the total weight of the composition excluding the weight of any liquid carrier).

In a fourteenth aspect, the disclosure provides solid-state compositions comprising any compounds of the first or second aspects, wherein the compounds of the first or second aspects make up at least 20% by weight of the solid-state compositions, based on the total weight of composition.

In a fifteenth aspect, the disclosure provides ingestible compositions comprising any compounds of the first or second aspects, wherein the concentration of the compounds of the first or second aspects in the ingestible compositions is no more than 200 ppm. In some embodiments, the ingestible composition is not a naturally occurring composition.

In a sixteenth aspect, the disclosure provides ingestible compositions comprising any compounds of the first or second aspects and a sweetener. In some embodiments, the sweetener is a caloric sweetener, such as sucrose, fructose, glucose, xylitol, erythritol, or combinations thereof. In some embodiments, the sweetener is a non-caloric sweetener, such as a steviol glycoside, a mogroside, aspartame, sucralose, acesulfame K, saccharin, or any combinations thereof. In some embodiments, the ingestible composition comprises one or more high-intensity sweeteners.

In an seventeenth aspect, the disclosure provides a concentrated sweetening composition comprising any compounds of the first or second aspects and a sweetener.

In an eighteenth aspect, the disclosure provides flavored products comprising any compositions of the preceding five aspects. In some embodiment, the flavored products are beverage products, such as soda, flavored water, tea, and the like. In some other embodiments, the flavored products are food products, such as yogurt.

In a nineteenth aspect, the disclosure provides methods of making compounds of the first or second aspects, comprising: providing a licorice polyglucuronide; and contacting the licorice polyglucuronide with an enzyme to remove one or more glucuronide moieties to form a from a licorice monoglucuronide. In some embodiments, further functionalization is carried out.

Further aspects, and embodiments thereof, are set forth below in the Detailed Description, the Drawings, the Abstract, and the Claims.

### BRIEF DESCRIPTION OF THE DRAWINGS

The following drawings are provided for purposes of illustrating various embodiments of the compositions and methods disclosed herein. The drawings are provided for illustrative purposes only, and are not intended to describe any preferred compositions or preferred methods, or to serve as a source of any limitations on the scope of the claimed inventions.

FIG. 1 shows a chemical formula that represents non-limiting examples of compounds (or salts thereof) disclosed herein, wherein: R¹ is a hydrogen atom, hydroxyl, or an optionally substituted alkoxy; R² is a hydrogen atom, hydroxyl, an optionally substituted alkoxy, an alkoxycarbonyloxy, or a glucosyloxy; R³ is a hydrogen atom, a glucuronyl, a glucuronyl ester, or a glucosyl; one of R⁴ and R⁵ is a methyl group, and the other is —C(O)—O—R^{4A}; R^{4A} is a hydrogen atom, a C₁₋₆ alkyl, a glucuronyl moiety, a C₁₋₆ alkyl glucuronyl ester moiety, or a glucosyl moiety; R⁶ is a hydrogen atom or C₁₋₆ alkyl; and X¹ is —CH₂— or >C═O.

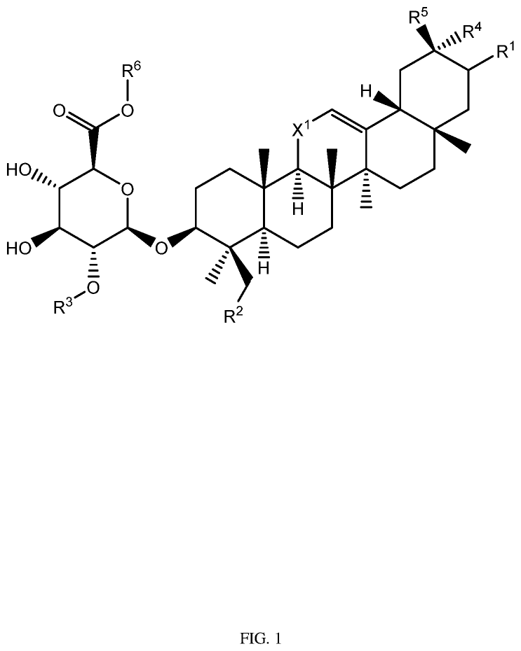

### DETAILED DESCRIPTION

The following Detailed Description sets forth various aspects and embodiments provided herein. The description is to be read from the perspective of the person of ordinary skill in the relevant art. Therefore, information that is well known to such ordinarily skilled artisans is not necessarily included.

### Definitions

The following terms and phrases have the meanings indicated below, unless otherwise provided herein. This disclosure may employ other terms and phrases not expressly defined herein. Such other terms and phrases have the meanings that they would possess within the context of this disclosure to those of ordinary skill in the art. In some instances, a term or phrase may be defined in the singular or plural. In such instances, it is understood that any term in the singular may include its plural counterpart and vice versa, unless expressly indicated to the contrary

As used herein, “solvate” means a compound formed by the interaction of one or more solvent molecules and one or more compounds described herein. In some embodiments, the solvates are ingestibly acceptable solvates, such as hydrates.

As used herein, “Cₐ to C_{b}” or “C_{a-b}” in which “a” and “b” are integers, refer to the number of carbon atoms in the specified group. That is, the group can contain from “a” to “b”, inclusive, carbon atoms. Thus, for example, a “C₁ to C₄ alkyl” or “C₁₋₄ alkyl” group refers to all alkyl groups having from 1 to 4 carbons, that is, CH₃—, CH₃CH₂—, CH₃CH₂CH₂—, (CH₃)₂CH—, CH₃CH₂CH₂CH₂—, CH₃CH₂CH(CH₃)— and (CH₃)₃C—.

As used herein, “alkyl” means a straight or branched hydrocarbon chain that is fully saturated (i.e., contains no double or triple bonds). In some embodiments, an alkyl group has 1 to 20 carbon atoms (whenever it appears herein, a numerical range such as “1 to 20” refers to each integer in the given range; e.g., “1 to 20 carbon atoms” means that the alkyl group may consist of 1 carbon atom, 2 carbon atoms, 3 carbon atoms, etc., up to and including 20 carbon atoms, although the present definition also covers the occurrence of the term “alkyl” where no numerical range is designated). The alkyl group may also be a medium size alkyl having 1 to 9 carbon atoms. The alkyl group could also be a lower alkyl having 1 to 4 carbon atoms. The alkyl group may be designated as “C₁₋₄ alkyl” or similar designations. By way of example only, “C14 alkyl” indicates that there are one to four carbon atoms in the alkyl chain, i.e., the alkyl chain is selected from the group consisting of methyl, ethyl, propyl, iso-propyl, n-butyl, iso-butyl, sec-butyl, and t-butyl. Typical alkyl groups include, but are in no way limited to, methyl, ethyl, propyl, isopropyl, butyl, isobutyl, tertiary butyl, pentyl, hexyl, and the like. Unless indicated to the contrary, the term “alkyl” refers to a group that is not further substituted.

As used herein, “alkoxy” means a moiety of the formula —OR wherein R is an alkyl, as is defined above, such as “C₁₋₉ alkoxy”, including but not limited to methoxy, ethoxy, n-propoxy, 1-methylethoxy (isopropoxy), n-butoxy, iso-butoxy, sec-butoxy, and tert-butoxy, and the like.

As used herein, a “glucosyl moiety” is a monovalent moiety in which one of the hydroxyl groups of glucose is replaced by a bond to another atom, functional group, or moiety. Unless otherwise specified, the glucose can have any suitable stereochemistry.

Thus, the term includes moieties having D stereochemistry, as well as moieties having L stereochemistry. Further, the term includes moieties having a stereochemistry, as well as moieties having β stereochemistry. The carbon atoms of the glucosyl moiety follow the conventional numbering, as shown below. The diagram is shown for β-D glucose, but applies in an analogous way to glucosyl moieties having α and/or L stereochemistry:

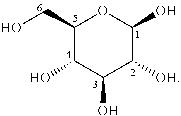 [SMILES: OC[C@H]1O[C@@H](O)[C@H](O)[C@@H](O)[C@@H]1O]

As used herein, a “glucuronyl moiety” is a monovalent moiety in which one of the hydroxyl groups of glucuronic acid is replaced by a bond to another atom, functional group, or moiety. Unless otherwise specified, the glucuronic acid can have any suitable stereochemistry. Thus, the term includes moieties having D stereochemistry, as well as moieties having L stereochemistry. Further, the term includes moieties having a stereochemistry, as well as moieties having β stereochemistry. The carbon atoms of the gluruconyl moiety follow the conventional numbering, as shown below. The diagram is shown for β-D glucuronic acid, but applies in an analogous way to glucuronyl moieties having α and/or L stereochemistry:

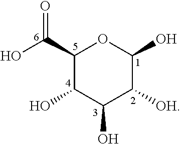 [SMILES: O=C(O)[C@H]1O[C@@H](O)[C@H](O)[C@@H](O)[C@@H]1O]

The disclosure refers to a “C₁₋₆ alkyl glucuronyl ester moiety,” which refers to a glucuronyl moiety (as defined in this paragraph) in which the carboxylic acid group of glucuronic acid has a C₁₋₆ alkyl group in place of the hydrogen atom of the carboxylic acid group. In any of the embodiments below, the C₁₋₆ alkyl moiety can have any suitable value, such as methyl, ethyl, isopropyl, propyl, butyl, pentyl, and the like. In some embodiments, the C₁₋₆ alkyl moiety is methyl. In some other embodiments, the C₁₋₆ alkyl moiety is ethyl.

It is to be understood that certain radical naming conventions can include either a mono-radical or a di-radical, depending on the context. For example, where a substituent requires two points of attachment to the rest of the molecule, it is understood that the substituent is a di-radical. For example, a substituent identified as alkyl that requires two points of attachment includes di-radicals such as —CH₂—, —CH₂CH₂—, —CH₂CH(CH₃)CH₂—, and the like.

Wherever a substituent is depicted as a di-radical (i.e., has two points of attachment to the rest of the molecule), it is to be understood that the substituent can be attached in any directional configuration unless otherwise indicated. Thus, for example, a substituent depicted as -AE- or

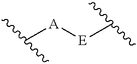

includes the substituent being oriented such that the A is attached at the leftmost attachment point of the molecule as well as the case in which A is attached at the rightmost attachment point of the molecule.

A “sweetener”, “sweet flavoring agent”, “sweet flavor entity”, or “sweet compound” herein refers to a compound or ingestibly acceptable salt thereof that elicits a detectable sweet flavor in a subject, e.g., a compound that activates a T1R2/T1R3 receptor in vitro.

As used herein, the singular forms “a,” “an,” and “the” include plural referents unless the context clearly dictates otherwise. For example, reference to “a substituent” encompasses a single substituent as well as two or more substituents, and the like. As used herein, “for example,” “for instance,” “such as,” or “including” are meant to introduce examples that further clarify more general subject matter. Unless otherwise expressly indicated, such examples are provided only as an aid for understanding embodiments illustrated in the present disclosure, and are not meant to be limiting in any fashion. Nor do these phrases indicate any kind of preference for the disclosed embodiment.

As used herein, “comprise” or “comprises” or “comprising” or “comprised of refer to groups that are open, meaning that the group can include additional members in addition to those expressly recited. For example, the phrase, “comprises A” means that A must be present, but that other members can be present too. The terms “include,” “have,” and “composed of” and their grammatical variants have the same meaning. In contrast, “consist of” or “consists of” or “consisting of” refer to groups that are closed. For example, the phrase “consists of A” means that A and only A is present.

As used herein, “optionally” means that the subsequently described event(s) may or may not occur. In some embodiments, the optional event does not occur. In some other embodiments, the optional event does occur one or more times.

As used herein, “or” is to be given its broadest reasonable interpretation, and is not to be limited to an either/or construction. Thus, the phrase “comprising A or B” means that A can be present and not B, or that B is present and not A, or that A and B are both present. Further, if A, for example, defines a class that can have multiple members, e.g., A₁ and A₂, then one or more members of the class can be present concurrently.

As used herein, certain substituents or linking groups having only a single atom may be referred to by the name of the atom. For example, in some cases, the substituent “—H” may be referred to as “hydrogen” or “a hydrogen atom,” the substituent “—F” may be referred to as “fluorine” or “a fluorine atom,” and the linking group “—O—” may be referred to as “oxygen” or “an oxygen atom.”

Points of attachment for groups are generally indicated by a terminal dash (—) or by an asterisk (*). For example, a group such as *—CH₂—CH₃ or —CH₂—CH₃ both represent an ethyl group.

Chemical structures are often shown using the “skeletal” format, such that carbon atoms are not explicitly shown, and hydrogen atoms attached to carbon atoms are omitted entirely. For example, the structure

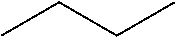

represents butane (i.e., n-butane). Furthermore, romatic groups, such as benzene, are represented by showing one of the contributing resonance structures. For example, the structure

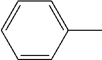

represents toluene.

Other terms are defined in other portions of this description, even though not included in this subsection.

### Licorice Glucuronides

In a first aspect, the disclosure provides flavor-modifying compounds, which are licorice glucuronides. As used herein, the term “licorice glucuronide” refers to a glucuronide of any of the terpene compounds (or derivatives thereof) found in licorice (Glycyrrhiza glabra). This includes any such compounds that exist naturally in the plant as particular glucuronides, as well as glucuronide derivatives of terpenes that exist in the plant in other forms (e.g., as free terpenes, or as different-order glucuronides of the same). Thus, the term “licorice glucuronide” does not imply that such compounds are necessarily extracted from licorice, or that they are derived from compounds that are extracted from licorice. Thus, while such compounds can be extracted from licorice or derived from compounds extracted from licorice, they can also be synthesized using typical organic reactions, enzyme-catalyzed transformations, or any combination thereof. For example, in some embodiments, “licorice glucuronides” are synthesized by a process that comprises in vitro enzyme-catalyzed reactions.

In some embodiments, the licorice glucuronides are licorice monoglucuronides. Such compounds may be extracted from licorice, or may be derived from by other means. For example, in some embodiments, licorice monoglucuronides are synthesized via the hydrolysis of licorice diglucuronides (e.g., through the removal of one glucuronide moiety from glycyrrhizin by hydrolysis). In some embodiments, the licorice monoglucuronides are licorice terpene monoglucuronides, such as monoglucuronides of terpene compounds present in licorice (or derivatives thereof). In some further embodiments, the licorice monoglucuronides are licorice triterpene monoglucuronides, such as monoglucuronides of triterpene compounds present in licorice (or derivatives thereof), such as monoglucuronides of glycyrrhetinic acid, or derivatives thereof.

Table 1 sets forth various examples of licorice glucuronides. In some embodiments, the licorice glucuronides comprise Compound 101. In some embodiments, the licorice glucuronides comprise Compound 102. In some embodiments, the licorice glucuronides comprise Compound 103. In some embodiments, the licorice glucuronides comprise Compound 104. In some embodiments, the licorice glucuronides comprise Compound 105. In some embodiments, the licorice glucuronides comprise Compound 106. In some embodiments, the licorice glucuronides comprise Compound 107.

TABLE 1

| No. | Structure |
|---|---|
| 101 | 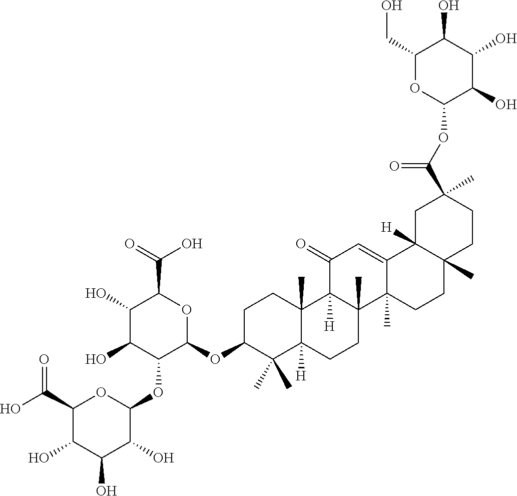 Licoricesaponin A₃ |
| 102 | 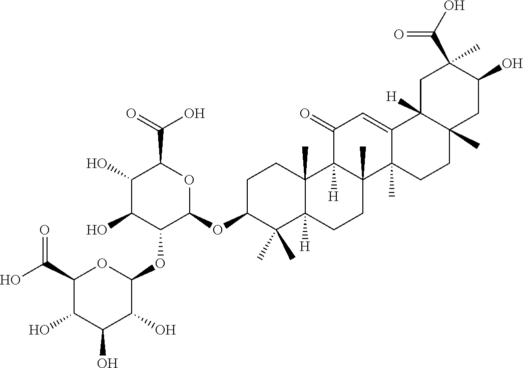 β-Macedonoside A |
| 103 | 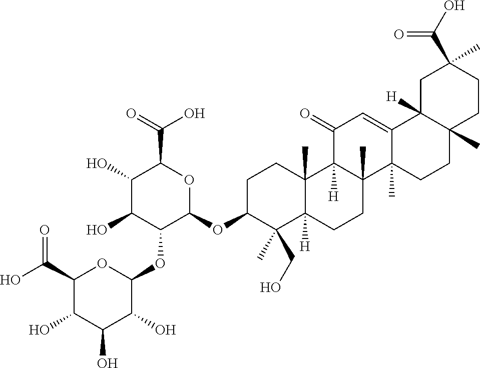 Licoricesaponin G₂ |
| 104 | 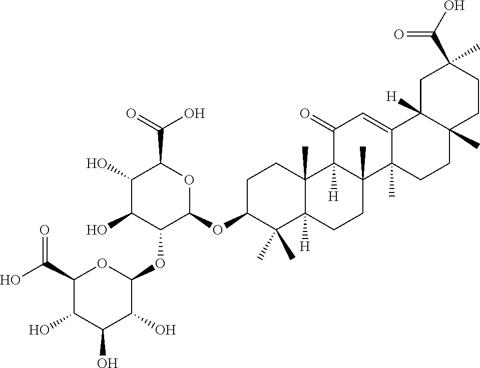 β-Glycyrrhizin |
| 105 | 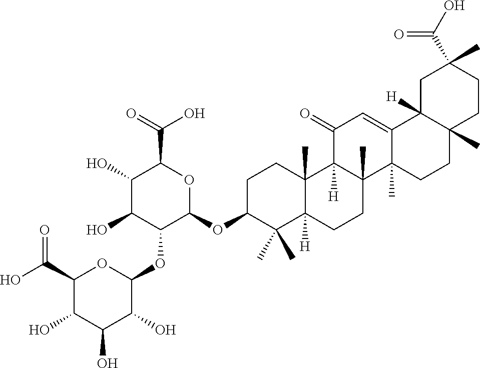 α-Glycyrrhizin (Licoricesaponin H₂) |
| 106 | 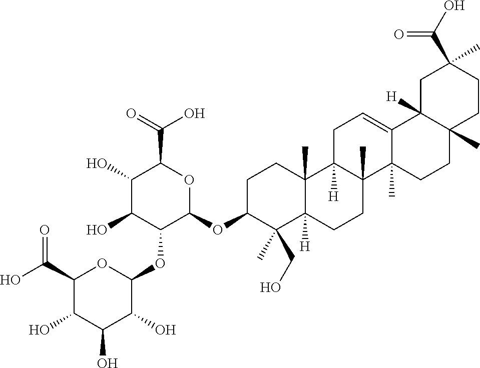 Licoricesaponin J₂ |
| 107 | 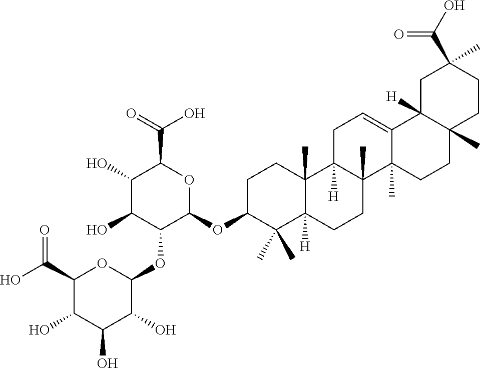 Licoricesaponin B₂ |
| 108 | 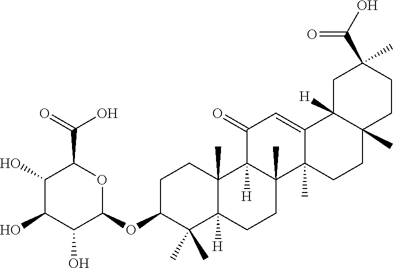 β-Glycyrrhizin Monoglucuronate (Glycyrrhetic acid 3-O-glucuronide) |
| 109 | 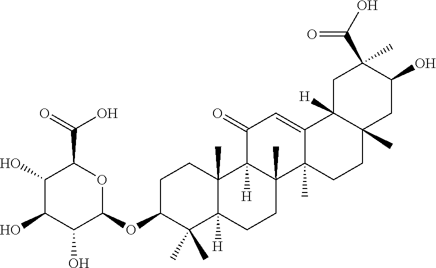 β-Macedonoside A Monoglucuronate |
| 110 | 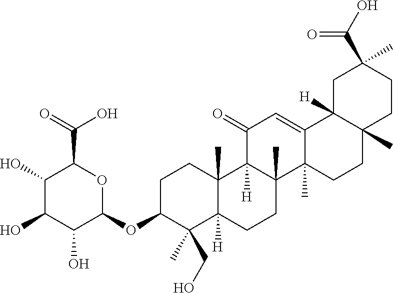 Licoricesaponin G₂ monoglucuronide |
| 111 | 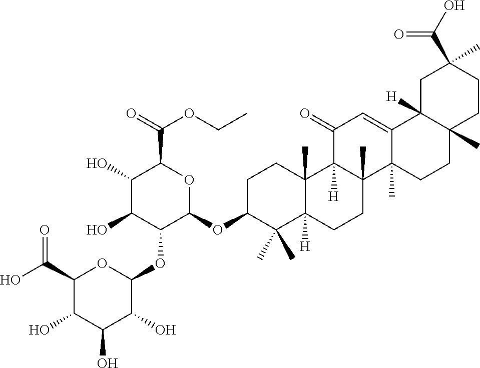 β-Glycyrrhizin ethyl ester I |
| 112 | 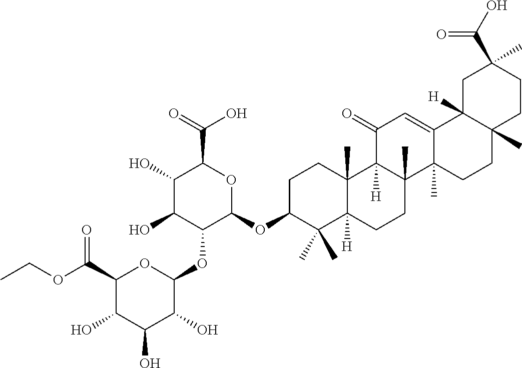 β-Glycyrrhizin ethyl ester II |

In a second aspect, the disclosure provides flavor-modifying compounds, which are compounds of formula (I):

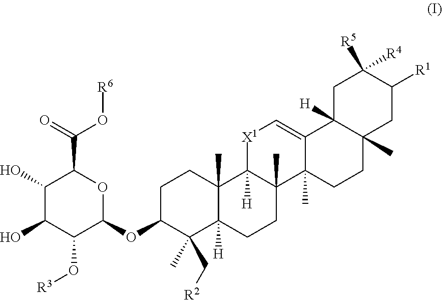

or salts thereof, wherein:

R¹ is a hydrogen atom, —OH, or —O—R^{1A};

R^{1A} is C₁₋₆ alkyl, which is optionally substituted one or more times by substituents selected from the group consisting of —OH and C₁₋₆ alkoxy;

R² is a hydrogen atom, —OH, or —O—R^{2A};

R^{2A} is C₁₋₆ alkyl, which is optionally substituted one or more times by substituents selected from the group consisting of —OH and C₁₋₆ alkoxy; or R^{2A} is —C(O)—(C₁₋₆ alkyl) or a glucosyl moiety;

R³ is a hydrogen atom, a glucuronyl moiety, a C₁₋₆ alkyl glucuronyl ester moiety, or a glucosyl moiety;

one of R⁴ and R⁵ is a methyl group, and the other is —C(O)—O—R^{4A};

R^{4A} is a hydrogen atom, C₁₋₆ alkyl, a glucuronyl moiety, a C₁₋₆ alkyl glucuronyl ester moiety, or a glucosyl moiety;

R⁶ is a hydrogen atom or C₁₋₆ alkyl; and

X¹ is —CH₂— or >C═O.

The variable R¹ can have any suitable value, according to the embodiments set forth above. In some embodiments, R¹ is a hydrogen atom. In some other embodiments, R¹ is an —OH moiety. In some other embodiments, R¹ is —O—R^{1A}. When R¹ is not a hydrogen atom, the point of attachment is a stereocenter, and the flat connection indicates that R¹ may be connected in a way such that the configuration around the stereocenter is either an R or an S configuration. In some such embodiments, R¹ is connected in a way such that the configuration around the stereocenter is an R configuration. In some other such embodiments, R¹ is connected in a way such that the configuration around the stereocenter is an S configuration. R^{1A} can have any suitable value according to the embodiments set forth above. In some embodiments, R^{1A} is an unsubstituted C₁₋₆ alkyl, such as methyl, ethyl, or isopropyl. In some further such embodiments, R^{1A} is methyl.

The variable R² can have any suitable value, according to the embodiments set forth above. In some embodiments of any of the above embodiments, R² is a hydrogen atom. In some other embodiments of any of the above embodiments, R² is an —OH moiety. In some other embodiments of any of the above embodiments, R² is —O—R^{2A}, R^{2A} can have any suitable value according to the embodiments set forth above. In some embodiments, R^{2A} is an unsubstituted C₁₋₆ alkyl, such as methyl, ethyl, or isopropyl. In some further such embodiments, R^{2A} is methyl.

The variable R³ can have any suitable value, according to the embodiments set forth above. In some embodiments of any of the above embodiments, R³ is a hydrogen atom. In some other embodiments of any of the above embodiments, R³ is a glucuronyl moiety. In some such embodiments, the glucuronyl moiety of R³ connects via an ether linkage, with the attachment to the glucuronyl moiety of R³ occurring at the 1-position carbon, the 2-position carbon, the 3-position carbon, or the 4-position carbon of said moiety. In some further such embodiments, the glucuronyl moiety of R³ connects via an ether linkage, with the attachment to the glucuronyl moiety of R³ occurring at the 1-position carbon. In some other embodiments of any of the above embodiments, R³ is a C₁₋₆ glucuronyl ester moiety. In some such embodiments, the C₁₋₆ glucuronyl ester moiety of R³ connects via linkage glycosidic bond, with the attachment to the C₁₋₆ glucuronyl ester moiety of R³ occurring at the 1-position carbon, the 2-position carbon, the 3-position carbon, or the 4-position carbon of said moiety. In some further such embodiments, the C₁₋₆ glucuronyl ester moiety of R³ connects via linkage glycosidic bond, with the attachment to the C₁₋₆ glucuronyl ester moiety of R³ occurring at the 1-position carbon. In some other embodiments of any of the above embodiments, R³ is a glucosyl moiety. In some such embodiments, the glucosyl moiety of R³ connects via an ether linkage, with the attachment to the glucosyl moiety of R³ occurring at the 1-position carbon, the 2-position carbon, the 3-position carbon, or the 4-position carbon of said moiety. In some further such embodiments, the glucosyl moiety of R³ connects via an ether linkage, with the attachment to the glucuronyl moiety of R³ occurring at the 1-position carbon.

The variables R⁴ and R⁵ can have any suitable value, according to the embodiments set forth above. In some embodiments of any of the above embodiments, R⁴ is methyl and R⁵ is —C(O)—O—R^{4A}. In some other embodiments of any of the above embodiments, R⁵ is methyl and R⁴ is —C(O)—O—R^{4A}. Regardless of whether —C(O)—O—R^{4A} is R⁴ or R⁵, the variable R^{4A} can have any suitable value, according to the embodiments set forth above. In some embodiments of any of the above embodiments, R^{4A} is a hydrogen atom. In some other embodiments of any of the above embodiments, R^{4A} is a glucuronyl moiety. In some such embodiments, the glucuronyl moiety of R^{4A} connects via an ether linkage, with the attachment to the glucuronyl moiety of R^{4A} occurring at the 1-position carbon, the 2-position carbon, the 3-position carbon, or the 4-position carbon of said moiety. In some further such embodiments, the glucuronyl moiety of R^{4A} connects via an ether linkage, with the attachment to the glucuronyl moiety of R^{4A} occurring at the 1-position carbon. In some other embodiments of any of the above embodiments, R^{4A} is a glucosyl moiety. In some such embodiments, the glucosyl moiety of R^{4A} connects via an ether linkage, with the attachment to the glucosyl moiety of R^{4A} occurring at the 1-position carbon, the 2-position carbon, the 3-position carbon, or the 4-position carbon of said moiety. In some further such embodiments, the glucosyl moiety of R^{4A} connects via an ether linkage, with the attachment to the glucuronyl moiety of R^{4A} occurring at the 1-position carbon. In some other embodiments, R^{4A} is C₁₋₆ alkyl, such as methyl, ethyl, isopropyl, propyl, butyl, and the like. In some such embodiments, R^{4A} is methyl or ethyl. In some further such embodiments, R^{4A} is methyl. In some other embodiments, R^{4A} is ethyl.

The variable R⁶ can have any suitable value, according to the embodiments set forth above. In some embodiments, R⁶ is a hydrogen atom. In some other embodiments, R⁶ is C₁₋₆ alkyl, such as methyl, ethyl, isopropyl, propyl, butyl, and the like. In some such embodiments, R⁶ is methyl or ethyl. In some further such embodiments, R⁶ is methyl. In some other embodiments, R⁶ is ethyl.

The variable X¹ can have any suitable value, according to the embodiments set forth above. In some embodiments of any of the above embodiments, X¹ is —CH₂— (a methylene group). In some other embodiments of any of the above embodiments, X¹ is >C═O (a carbonyl group).

In some embodiments of any of the above embodiments, the flavor-modifying compounds are compounds of formula (Ia):

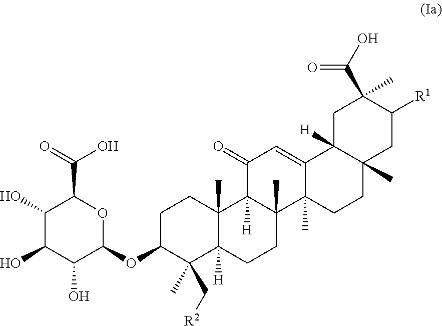

or salts thereof, wherein:

R¹ is a hydrogen atom, —OH, or —O—R^{1A};

R^{1A} is C₁₋₆ alkyl, which is optionally substituted one or more times by substituents selected from the group consisting of —OH and C₁₋₆ alkoxy;

R² is a hydrogen atom, —OH, or —O—R^{2A}; and

R^{2A} is C₁₋₆ alkyl, which is optionally substituted one or more times by substituents selected from the group consisting of —OH and C₁₋₆ alkoxy. In some further embodiments thereof, R¹ is —OH and R² is a hydrogen atom. In some other embodiments thereof, R¹ is a hydrogen atom and R² is —OH. In some other embodiments thereof, R¹ is —OH and R² is —OH.

Where the compounds disclosed herein have at least one chiral center, they may exist as individual enantiomers and diastereomers or as mixtures of such isomers. In some embodiments in connection with the second aspect, the sweet-enhancing compound has substantial enantiomeric purity.

Separation of the individual isomers or selective synthesis of the individual isomers is accomplished by application of various methods which are well known to practitioners in the art. Unless otherwise indicated (e.g., where the stereochemistry of a chiral center is explicitly shown), all such isomers and mixtures thereof are included in the scope of the compounds disclosed herein. Furthermore, compounds disclosed herein may exist in one or more crystalline or amorphous forms. Unless otherwise indicated, all such forms are included in the scope of the compounds disclosed herein including any polymorphic forms. In addition, some of the compounds disclosed herein may form solvates with water (i.e., hydrates) or common organic solvents. Unless otherwise indicated, such solvates are included in the scope of the compounds disclosed herein.

The skilled artisan will recognize that some structures described herein may be resonance forms or tautomers of compounds that may be fairly represented by other chemical structures, even when kinetically; the artisan recognizes that such structures may only represent a very small portion of a sample of such compound(s). Such compounds are considered within the scope of the structures depicted, though such resonance forms or tautomers are not represented herein.

Isotopes may be present in the compounds described. Each chemical element as represented in a compound structure may include any isotope of said element. For example, in a compound structure a hydrogen atom may be explicitly disclosed or understood to be present in the compound. At any position of the compound that a hydrogen atom may be present, the hydrogen atom can be any isotope of hydrogen, including but not limited to hydrogen-1 (protium) and hydrogen-2 (deuterium). Thus, reference herein to a compound encompasses all potential isotopic forms unless the context clearly dictates otherwise.

In some embodiments, the compounds disclosed herein are capable of forming acid and/or base salts by virtue of the presence of amino and/or carboxyl groups or groups similar thereto. Physiologically acceptable acid addition salts can be formed with inorganic acids and organic acids. Inorganic acids from which salts can be derived include, for example, hydrochloric acid, hydrobromic acid, sulfuric acid, nitric acid, phosphoric acid, and the like.

Organic acids from which salts can be derived include, for example, acetic acid, propionic acid, glycolic acid, pyruvic acid, oxalic acid, maleic acid, malonic acid, succinic acid, fumaric acid, tartaric acid, citric acid, benzoic acid, cinnamic acid, mandelic acid, methanesulfonic acid, ethanesulfonic acid, p-toluenesulfonic acid, salicylic acid, and the like. Physiologically acceptable salts can be formed using inorganic and organic bases. Inorganic bases from which salts can be derived include, for example, bases that contain sodium, potassium, lithium, ammonium, calcium, magnesium, iron, zinc, copper, manganese, aluminum, and the like; particularly preferred are the ammonium, potassium, sodium, calcium and magnesium salts. In some embodiments, treatment of the compounds disclosed herein with an inorganic base results in loss of a labile hydrogen from the compound to afford the salt form including an inorganic cation such as Li⁺, Na⁺, K⁺, Mg²⁺ and Ca²⁺ and the like. Organic bases from which salts can be derived include, for example, primary, secondary, and tertiary amines, substituted amines including naturally occurring substituted amines, cyclic amines, basic ion exchange resins, and the like, specifically such as isopropylamine, trimethylamine, diethylamine, triethylamine, tripropylamine, and ethanolamine. In some embodiments, the salts are comestibly acceptable salts, which are salts suitable for inclusion in comestible food and/or beverage products.

### Uses and Methods

In another aspect, the disclosure provides uses of any flavor-modifying compounds of the foregoing aspects, including any embodiments or combination of embodiments thereof, as set forth above. In certain related aspects, the disclosure provides uses of any flavor- modifying compounds of the foregoing aspects, including any embodiments or combination of embodiments thereof, as set forth above, to enhance the sweetness of an ingestible composition. In some embodiments thereof, the ingestible composition comprises a sweetener, such as a caloric sweetener. In certain other related aspects, the disclosure provides uses of any flavor-modifying compounds of the foregoing aspects, including any embodiments or combination of embodiments thereof, as set forth above, to reduce the sourness of an ingestible composition. In another related aspect, the disclosure provides uses of any flavor-modifying compounds of the foregoing aspects, including any embodiments or combination of embodiments thereof, as set forth above, to reduce the bitterness of an ingestible composition. In certain other related aspects, the disclosure provides uses of any flavor-modifying compounds of the foregoing aspects, including any embodiments or combination of embodiments thereof, as set forth above, in the manufacture of an ingestible composition to enhance the sweetness of the ingestible composition. In some embodiments thereof, the ingestible composition comprises a caloric sweetener. In another related aspect, the disclosure provides uses of any flavor-modifying compounds of foregoing aspects, including any embodiments or combination of embodiments thereof, as set forth above, in the manufacture of an ingestible composition to reduce the sourness of the ingestible composition. In another related aspect, the disclosure provides uses of any flavor-modifying compounds of the foregoing aspects, including any embodiments or combination of embodiments thereof, as set forth above, in the manufacture of an ingestible composition to reduce the bitterness of the ingestible composition.

The disclosure also provides methods that correspond to each of the foregoing uses. Thus, in certain related aspects, the disclosure provides methods of enhancing the sweetness of an ingestible composition, comprising introducing an amount (such as a sweetness- enhancing effective amount) of any flavor-modifying compounds of foregoing aspects, including any embodiments or combination of embodiments thereof, as set forth above, to the ingestible composition. In some other related aspects, the disclosure provides methods of reducing the sourness of an ingestible composition, comprising introducing an amount (such as a sourness-reducing effective amount) of any flavor-modifying compounds of foregoing aspects, including any embodiments or combination of embodiments thereof, as set forth above, to the ingestible composition. In some other related aspects, the disclosure provides methods of reducing the bitterness of an ingestible composition, comprising introducing an amount (such as a bitterness-reducing effective amount) of any flavor-modifying compounds of foregoing aspects, including any embodiments or combination of embodiments thereof, as set forth above, to the ingestible composition.

The foregoing uses and methods generally involve the use of the flavor-modifying compounds in a composition containing one or more additional ingredients. For example, in at least one aspect, the disclosure provides compositions comprising any flavor-modifying compounds of the foregoing aspects, including any embodiments or combination of embodiments thereof, as set forth above, wherein the flavor-modifying compounds make up at least 50% by weight of the compositions on a dry weight basis (e.g., based on the total weight of the composition excluding the weight of any liquid carrier). In a related aspect, the disclosure provides solid-state compositions comprising any flavor-modifying compounds of the foregoing aspects, including any embodiments or combination of embodiments thereof, as set forth above, wherein the flavor-modifying compounds make up at least 50% by weight of the solid-state compositions, based on the total weight of composition. In another related aspect, the disclosure provides ingestible compositions comprising flavor-modifying compounds of the foregoing aspects, including any embodiments or combination of embodiments thereof, as set forth above, wherein the concentration of the flavor-modifying compounds in the ingestible compositions is no more than 200 ppm. In another related aspect, the disclosure provides ingestible compositions comprising any flavor-modifying compounds of the foregoing aspects, including any embodiments or combination of embodiments thereof, as set forth above, wherein the ingestible compositions comprise a caloric sweetener, such as sucrose, fructose, xylitol, erythritol, or combinations thereof. In another related aspect, the disclosure provides a concentrated sweetening composition comprising any flavor-modifying compounds of the foregoing aspects, including any embodiments or combination of embodiments thereof, as set forth above, and a sweetener.

In certain embodiments of any aspects and embodiments set forth herein that refer to an ingestible composition, the ingestible composition is a non-naturally-occurring product, such as a composition specifically manufactured for the production of a flavored product, such as food or beverage product.

In general, compounds as disclosed and described herein, individually or in combination, can be provided in a composition, such as an ingestible composition. In one embodiment, compounds as disclosed and described herein, individually or in combination, can impart a more sugar-like temporal profile or flavor profile to a sweetener composition by combining one or more of the compounds as disclosed and described herein with one or more sweeteners in the sweetener composition. In another embodiment, compounds as disclosed and described herein, individually or in combination, can increase or enhance the sweet taste of a composition by contacting the composition thereof with the compounds as disclosed and described herein to form a modified composition.

Thus, in some embodiments, the compositions set forth in any of the foregoing aspects (including in any uses or methods), comprise a flavor-modifying compound and a sweetener. In some embodiments, the composition further comprises a vehicle. In some embodiments, the vehicle is water. In some embodiments, the flavor-modifying compound is present at a concentration at or below its sweetness recognition threshold.

For example, in some embodiments, the sweetener is present in an amount from about 0.1% to about 12% by weight. In some embodiments, the sweetener is present in an amount from about 0.2% to about 10% by weight. In some embodiments, the sweetener is present in an amount from about 0.3% to about 8% by weight. In some embodiments, the sweetener is present in an amount from about 0.4% to about 6% by weight. In some embodiments, the sweetener is present in an amount from about 0.5% to about 5% by weight. In some embodiments, the sweetener is present in an amount from about 1% to about 2% by weight. In some embodiments, the sweetener is present in an amount from about 0.1% to about 5% by weight. In some embodiments, the sweetener is present in an amount from about 0.1% to about 4% by weight. In some embodiments, the sweetener is present in an amount from about 0.1% to about 3% by weight. In some embodiments, the sweetener is present in an amount from about 0.1% to about 2% by weight. In some embodiments, the sweetener is present in an amount from about 0.1% to about 1% by weight. In some embodiments, the sweetener is present in an amount from about 0.1% to about 0.5% by weight. In some embodiments, the sweetener is present in an amount from about 0.5% to about 10% by weight. In some embodiments, the sweetener is present in an amount from about 2% to about 8% by weight. In some further embodiments of the embodiments set forth in this paragraph, the sweetener is sucrose, fructose, glucose, xylitol, erythritol, or combinations thereof.

In some other embodiments, the sweetener is present in an amount from 10 ppm to 1000 ppm. In some embodiments, the sweetener is present in an amount from 20 ppm to 800 ppm. In some embodiments, the sweetener is present in an amount from 30 ppm to 600 ppm. In some embodiments, the sweetener is present in an amount from 40 ppm to 500 ppm. In some embodiments, the sweetener is present in an amount from 50 ppm to 400 ppm. In some embodiments, the sweetener is present in an amount from 50 ppm to 300 ppm. In some embodiments, the sweetener is present in an amount from 50 ppm to 200 ppm. In some embodiments, the sweetener is present in an amount from 50 ppm to 150 ppm. In some further embodiments of the embodiments set forth in this paragraph, the sweetener is a steviol glycoside, a mogroside, a derivative of either of the foregoing, such as glycoside derivatives (e.g., glucosylates), or any combination thereof.

The compositions can include any suitable sweeteners or combination of sweeteners. In some embodiments, the sweetener is a common saccharide sweeteners, such as sucrose, fructose, glucose, and sweetener compositions comprising natural sugars, such as corn syrup (including high fructose corn syrup) or other syrups or sweetener concentrates derived from natural fruit and vegetable sources. In some embodiments, the sweetener is sucrose, fructose, or a combination thereof. In some embodiments, the sweetener is sucrose. In some other embodiments, the sweetener is selected from rare natural sugars including D-allose, D-psicose, L-ribose, D-tagatose, L-glucose, L-fucose, L-arbinose, D-turanose, and D-leucrose. In some embodiments, the sweetener is selected from semi-synthetic “sugar alcohol” sweeteners such as erythritol, isomalt, lactitol, mannitol, sorbitol, xylitol, maltodextrin, and the like. In some embodiments, the sweetener is selected from artificial sweeteners such as aspartame, saccharin, acesulfame-K, cyclamate, sucralose, and alitame. In some embodiments, the sweetener is selected from the group consisting of cyclamic acid, mogroside, tagatose, maltose, galactose, mannose, sucrose, fructose, lactose, neotame and other aspartame derivatives, glucose, D-tryptophan, glycine, maltitol, lactitol, isomalt, hydrogenated glucose syrup (HGS), hydrogenated starch hydrolyzate (HSH), stevioside, rebaudioside A, other sweet Stevia-based glycosides, chemically modified steviol glycosides (such as glucosylated steviol glycosides), mogrosides, chemically modified mogrosides (such as glucosylated mogrosides), carrelame and other guanidine-based sweeteners. In some embodiments, the sweetener is a combination of two or more of the sweeteners set forth in this paragraph. In some embodiments, the sweetener may combinations of two, three, four or five sweeteners as disclosed herein. In some embodiments, the sweetener may be a sugar. In some embodiments, the sweetener may be a combination of one or more sugars and other natural and artificial sweeteners. In some embodiments, the sweetener is a sugar. In some embodiments, the sugar is cane sugar. In some embodiments, the sugar is beet sugar. In some embodiments, the sugar may be sucrose, fructose, glucose or combinations thereof. In some embodiments, the sugar may be sucrose. In some embodiments, the sugar may be a combination of fructose and glucose.

The sweetener can also include, for example, sweetener compositions comprising one or more natural or synthetic carbohydrate, such as corn syrup, high fructose corn syrup, high maltose corn syrup, glucose syrup, sucralose syrup, hydrogenated glucose syrup (HGS), hydrogenated starch hydrolyzate (HSH), or other syrups or sweetener concentrates derived from natural fruit and vegetable sources, or semi-synthetic “sugar alcohol” sweeteners such as polyols. Non-limiting examples of polyols in some embodiments include erythritol, maltitol, mannitol, sorbitol, lactitol, xylitol, isomalt, propylene glycol, glycerol (glycerin), threitol, galactitol, palatinose, reduced isomalto-oligosaccharides, reduced xylo-oligosaccharides, reduced gentio-oligosaccharides, reduced maltose syrup, reduced glucose syrup, isomaltulose, maltodextrin, and the like, and sugar alcohols or any other carbohydrates or combinations thereof capable of being reduced which do not adversely affect taste.

The sweetener may be a natural or synthetic sweetener that includes, but is not limited to, agave inulin, agave nectar, agave syrup, amazake, brazzein, brown rice syrup, coconut crystals, coconut sugars, coconut syrup, date sugar, fructans (also referred to as inulin fiber, fructo-oligosaccharides, or oligo-fructose), green stevia powder, stevia rebaudiana, rebaudioside A, rebaudioside B, rebaudioside C, rebaudioside D, rebaudioside E, rebaudioside F, rebaudioside I, rebaudioside H, rebaudioside L, rebaudioside K, rebaudioside J, rebaudioside N, rebaudioside O, rebaudioside M and other sweet stevia-based glycosides, stevioside, stevioside extracts, honey, Jerusalem artichoke syrup, licorice root, luo han guo (fruit, powder, or extracts), lucuma (fruit, powder, or extracts), maple sap (including, for example, sap extracted from Acer saccharum, Acer nigrum, Acer rubrum, Acer saccharinum, Acer platanoides, Acer negundo, Acer macrophyllum, Acer grandidentatum, Acer glabrum, Acer mono), maple syrup, maple sugar, walnut sap (including, for example, sap extracted from Juglans cinerea, Juglans nigra, Juglans ailatifolia, Juglans regia), birch sap (including, for example, sap extracted from Betula papyrifera, Betula alleghaniensis, Betula lenta, Betula nigra, Betula populifolia, Betula pendula), sycamore sap (such as, for example, sap extracted from Platanus occidentalis), ironwood sap (such as, for example, sap extracted from Ostrya virginiana), mascobado, molasses (such as, for example, blackstrap molasses), molasses sugar, monatin, monellin, cane sugar (also referred to as natural sugar, unrefined cane sugar, or sucrose), palm sugar, panocha, piloncillo, rapadura, raw sugar, rice syrup, sorghum, sorghum syrup, cassava syrup (also referred to as tapioca syrup), thaumatin, yacon root, malt syrup, barley malt syrup, barley malt powder, beet sugar, cane sugar, crystalline juice crystals, caramel, carbitol, carob syrup, castor sugar, hydrogenated starch hydrolates, hydrolyzed can juice, hydrolyzed starch, invert sugar, anethole, arabinogalactan, arrope, syrup, P-4000, acesulfame potassium (also referred to as acesulfame K or ace-K), alitame (also referred to as aclame), advantame, aspartame, baiyunoside, neotame, benzamide derivatives, bernadame, canderel, carrelame and other guanidine-based sweeteners, vegetable fiber, corn sugar, coupling sugars, curculin, cyclamates, cyclocarioside I, demerara, dextran, dextrin, diastatic malt, dulcin, sucrol, valzin, dulcoside A, dulcoside B, emulin, enoxolone, maltodextrin, saccharin, estragole, ethyl maltol, glucin, gluconic acid, glucono-lactone, glucosamine, glucoronic acid, glycerol, glycine, glycyphillin, glycyrrhizin, glycyrrhetic acid monoglucuronide, golden sugar, yellow sugar, golden syrup, granulated sugar, gynostemma, hernandulcin, isomerized liquid sugars, jallab, chicory root dietary fiber, kynurenine derivatives (including N′-formyl-kynurenine, N′-acetyl-kynurenine, 6-chloro-kynurenine), galactitol, litesse, ligicane, lycasin, lugduname, guanidine, falernum, mabinlin I, mabinlin II, maltol, maltisorb, maltodextrin, maltotriol, mannosamine, miraculin, mizuame, mogrosides (including, for example, mogroside IV, mogroside V, and neomogroside), mukurozioside, nano sugar, naringin dihydrochalcone, neohesperidine dihydrochalcone, nib sugar, nigero- oligosaccharide, norbu, orgeat syrup, osladin, pekmez, pentadin, periandrin I, perillaldehyde, perillartine, petphyllum, phenylalanine, phlomisoside I, phlorodizin, phyllodulcin, polyglycitol syrups, polypodoside A, pterocaryoside A, pterocaryoside B, rebiana, refiners syrup, rub syrup, rubusoside, selligueain A, siamenoside I, siraitia grosvenorii, soybean oligosaccharide, Splenda, SRI oxime V, steviol glycoside, steviolbioside, stevioside, strogins 1, 2, and 4, sucronic acid, sucrononate, sugar, suosan, phloridzin, superaspartame, tetrasaccharide, threitol, treacle, trilobatin, D-tryptophan and derivatives (6-trifluoromethyl-tryptophan, 6-chloro-D-tryptophan), vanilla sugar, volemitol, birch syrup, aspartame-acesulfame, assugrin, aromedendrin-3-O-acetate, dihydrotamarixetin-3-O-acetate, and combinations or blends of any two or more thereof. In still other embodiments, the sweetener can be a chemically or enzymatically modified natural high potency sweetener. Modified natural high potency sweeteners include glycosylated natural high potency sweetener such as glucosyl-, galactosyl-, or fructosyl- derivatives containing 1-50 glycosidic residues. Glycosylated natural high potency sweeteners may be prepared by enzymatic transglycosylation reaction catalyzed by various enzymes possessing transglycosylating activity. In some embodiments, the modified sweetener can be substituted or unsubstituted.

Additional sweeteners also include combinations of any two or more of any of the aforementioned sweeteners. In some embodiments, the sweetener may comprise combinations of two, three, four or five sweeteners as disclosed herein. In some embodiments, the sweetener may be a sugar. In some embodiments, the sweetener may be a combination of one or more sugars and other natural and artificial sweeteners. In some embodiments, the sweetener is a caloric sweetener, such as sucrose, fructose, xylitol, erythritol, or combinations thereof. In some embodiments, the ingestible compositions are free (or, in some embodiments) substantially free of stevia-derived sweeteners, such as steviol glycosides, glucosylated steviol glycosides, or rebaudiosides. For example, in some embodiments, the ingestible compositions are either free of stevia-derived sweeteners or comprise stevia-derived sweeteners in a concentration of no more than 1000 ppm, or no more than 500 ppm, or no more than 200 ppm, or no more than 100 ppm, or no more than 50 ppm, or no more than 20 ppm, or no more than 10 ppm, or no more than 5 ppm, or no more than 3 ppm, or no more than 1 ppm.

The flavor-modifying compounds can be present in the ingestible compositions in any suitable amount. In some embodiments, the flavor-modifying compounds are present in an amount sufficient to enhance the taste (e.g., enhance the sweetness, reduce the sourness, or reduce the bitterness) of the compositions. Thus, in some embodiments, the ingestible composition comprises the flavor-modifying compound in a concentration no greater than 200 ppm, or no greater than 150 ppm, or no greater than 100 ppm, or no greater than 50 ppm, or no greater than 40 ppm, or no greater than 30 ppm, or no greater than 20 ppm. In some embodiments, the flavor-modifying compound is present in a minimum amount, such as 1 ppm or 5 ppm. Thus, in some embodiments, the ingestible composition comprises the flavor-modifying compound in a concentration ranging from 1 ppm to 200 ppm, or from 1 ppm to 150 ppm, or from 1 ppm to 100 ppm, or from 1 ppm to 50 ppm, or from 1 ppm to 40 ppm, or from 1 ppm to 30 ppm, or from 1 ppm to 20 ppm, or from 5 ppm to 200 ppm, or from 5 ppm to 150 ppm, or from 5 ppm to 100 ppm, or from 5 ppm to 90 ppm, or from 5 ppm to 80 ppm, or from 5 ppm to 70 ppm, or from 5 ppm to 60 ppm, or from 5 ppm to 50 ppm, or from 5 ppm to 40 ppm, or from 5 ppm to 30 ppm, or from 5 ppm to 20 ppm. In embodiments where a sweetener, such as sucrose or fructose, are present, the weight-to-weight ratio of sweetener to the flavor-modifying compound in the ingestible composition ranges from 1000:1 to 50000:1, or from 1000:1 to 10000:1, or from 2000:1 to 8000:1.

The flavor-modifying compounds can be combined in any suitable way. For example, in some embodiments, the ingestible composition or sweetener concentrate contains a single flavor-modifying compound of the embodiments set forth above, such as any one of the compounds identified above as Compound 101, Compound 102, Compound 103, Compound 104, Compound 105, Compound 106, Compound 107, Compound 108, Compound 109, Compound 110, Compound 111, or Compound 112. In some other embodiments, the ingestible composition or sweetener concentrate contains mixtures of two or more flavor-modifying compounds of the embodiments set forth above. Limiting examples of such mixtures include a mixture of Compound 109 and Compound 110. Such mixtures can include two or more flavor-modifying compounds of the embodiments set forth above in any suitable ratio. For example, in embodiments where the ingestible composition or sweetener concentrate contains a mixture of Compound 109 and Compound 110, the two compounds can be present in any suitable ratio relative to each other, such as w/w ratio ranging from 1:10 to 10:1, or 1:5 to 5:1, or from 1:3 to 3:1. The ingestible compositions or sweetener concentrates, in certain embodiments, also comprise any additional ingredients or combination of ingredients as are commonly used in food and beverage products, including, but not limited to:

acids, including, for example citric acid, phosphoric acid, ascorbic acid, sodium acid sulfate, lactic acid, or tartaric acid;

bitter ingredients, including, for example caffeine, quinine, green tea, catechins, polyphenols, green robusta coffee extract, green coffee extract, potassium chloride, menthol, or proteins (such as proteins and protein isolates derived from plants, algae, or fungi);

coloring agents, including, for example caramel color, Red #40, Yellow #5, Yellow #6, Blue #1, Red #3, purple carrot, black carrot juice, purple sweet potato, vegetable juice, fruit juice, beta carotene, turmeric curcumin, or titanium dioxide;

preservatives, including, for example sodium benzoate, potassium benzoate, potassium sorbate, sodium metabisulfate, sorbic acid, or benzoic acid;

antioxidants including, for example ascorbic acid, calcium disodium EDTA, alpha tocopherols, mixed tocopherols, rosemary extract, grape seed extract, resveratrol, or sodium hexametaphosphate;

vitamins or functional ingredients including, for example resveratrol, Co-Q10, omega 3 fatty acids, theanine, choline chloride (citocoline), fibersol, inulin (chicory root), taurine, panax ginseng extract, guanana extract, ginger extract, L-phenylalanine, L-carnitine, L-tartrate, D-glucoronolactone, inositol, bioflavonoids, Echinacea, ginko biloba, yerba mate, flax seed oil, garcinia cambogia rind extract, white tea extract, ribose, milk thistle extract, grape seed extract, pyrodixine HCl (vitamin B6), cyanoobalamin (vitamin B12), niacinamide (vitamin B3), biotin, calcium lactate, calcium pantothenate (pantothenic acid), calcium phosphate, calcium carbonate, chromium chloride, chromium polynicotinate, cupric sulfate, folic acid, ferric pyrophosphate, iron, magnesium lactate, magnesium carbonate, magnesium sulfate, monopotassium phosphate, monosodium phosphate, phosphorus, potassium iodide, potassium phosphate, riboflavin, sodium sulfate, sodium gluconate, sodium polyphosphate, sodium bicarbonate, thiamine mononitrate, vitamin D3, vitamin A palmitate, zinc gluconate, zinc lactate, or zinc sulphate;

clouding agents, including, for example ester gun, brominated vegetable oil (BYO), or sucrose acetate isobutyrate (SAIB);

buffers, including, for example sodium citrate, potassium citrate, or salt;

flavors, including, for example propylene glycol, ethyl alcohol, glycerine, gum Arabic (gum acacia), maltodextrin, modified corn starch, dextrose, natural flavor, natural flavor with other natural flavors (natural flavor WONF), natural and artificial flavors, artificial flavor, silicon dioxide, magnesium carbonate, or tricalcium phosphate; or

starches and stabilizers, including, for example pectin, xanthan gum, carboxylmethylcellulose (CMC), polysorbate 60, polysorbate 80, medium chain triglycerides, cellulose gel, cellulose gum, sodium caseinate, modified food starch, gum Arabic (gum acacia), inulin, or carrageenan.

The ingestible compositions or sweetener concentrates can have any suitable pH. In some embodiments, the flavor-modifying compounds enhance the sweetness of a sweetener under a broad range of pH, e.g., from lower pH to neutral pH. The lower and neutral pH includes, but is not limited to, a pH from about 2.5 to about 8.5; from about 3.0 to about 8.0; from about 3.5 to about 7.5; and from about 4.0 to about 7. In certain embodiments, compounds as disclosed and described herein, individually or in combination, can enhance the perceived sweetness of a fixed concentration of a sweetener in taste tests at a compound concentration of about 50 μM, 40 μM, 30 μM, 20 μM, or 10 μM at both low to neutral pH value. In certain embodiments, the enhancement factor of the compounds as disclosed and described herein, individually or in combination, at the lower pH is substantially similar to the enhancement factor of the compounds at neutral pH. Such consistent sweet enhancing property under a broad range of pH allow a broad use in a wide variety of foods and beverages of the compounds as disclosed and described herein, individually or in combination.

The ingestible compositions set forth according to any of the foregoing embodiments, also include, in certain embodiments, one or more additional flavor-modifying compounds, such as compounds that enhance sweetness (e.g., hesperetin, naringenin, glucosylated steviol glycosides, etc.), compounds that block bitterness, compounds that enhance umami, compounds that reduce sourness or licorice taste, compounds that enhance saltiness, compounds that enhance a cooling effect, or any combinations of the foregoing.

### Flavored Products and Concentrates

In certain aspects, the disclosure provides flavored products comprising any compositions of the preceding five aspects. In some embodiment, the flavored products are beverage products, such as soda, flavored water, tea, and the like. In some other embodiments, the flavored products are food products, such as yogurt.

In embodiments where the flavored product is a beverage, the beverage may be selected from the group consisting of enhanced sparkling beverages, colas, lemon-lime flavored sparkling beverages, orange flavored sparkling beverages, grape flavored sparkling beverages, strawberry flavored sparkling beverages, pineapple flavored sparkling beverages, ginger-ales, root beers, fruit juices, fruit-flavored juices, juice drinks, nectars, vegetable juices, vegetable-flavored juices, sports drinks, energy drinks, enhanced water drinks, enhanced water with vitamins, near water drinks, coconut waters, tea type drinks, coffees, cocoa drinks, beverages containing milk components, beverages containing cereal extracts and smoothies. In some embodiments, the beverage may be a soft drink.

In certain embodiments of any aspects and embodiments set forth herein that refer to an flavored product, the flavored product is a non-naturally-occurring product, such as a packaged food or beverage product.

Further non-limiting examples of food and beverage products or formulations include sweet coatings, frostings, or glazes for such products or any entity included in the Soup category, the Dried Processed Food category, the Beverage category, the Ready Meal category, the Canned or Preserved Food category, the Frozen Processed Food category, the Chilled Processed Food category, the Snack Food category, the Baked Goods category, the Confectionery category, the Dairy Product category, the Ice Cream category, the Meal Replacement category, the Pasta and Noodle category, and the Sauces, Dressings, Condiments category, the Baby Food category, and/or the Spreads category.

In general, the Soup category refers to canned/preserved, dehydrated, instant, chilled, UHT and frozen soup. For the purpose of this definition soup(s) means a food prepared from meat, poultry, fish, vegetables, grains, fruit and other ingredients, cooked in a liquid which may include visible pieces of some or all of these ingredients. It may be clear (as a broth) or thick (as a chowder), smooth, pureed or chunky, ready-to-serve, semi-condensed or condensed and may be served hot or cold, as a first course or as the main course of a meal or as a between meal snack (sipped like a beverage). Soup may be used as an ingredient for preparing other meal components and may range from broths (consommé) to sauces (cream or cheese-based soups).

The Dehydrated and Culinary Food Category usually means: (i) Cooking aid products such as: powders, granules, pastes, concentrated liquid products, including concentrated bouillon, bouillon and bouillon like products in pressed cubes, tablets or powder or granulated form, which are sold separately as a finished product or as an ingredient within a product, sauces and recipe mixes (regardless of technology); (ii) Meal solutions products such as: dehydrated and freeze dried soups, including dehydrated soup mixes, dehydrated instant soups, dehydrated ready-to-cook soups, dehydrated or ambient preparations of ready-made dishes, meals and single serve entrees including pasta, potato and rice dishes; and (iii) Meal embellishment products such as: condiments, marinades, salad dressings, salad toppings, dips, breading, batter mixes, shelf stable spreads, barbecue sauces, liquid recipe mixes, concentrates, sauces or sauce mixes, including recipe mixes for salad, sold as a finished product or as an ingredient within a product, whether dehydrated, liquid or frozen.

The Beverage category usually means beverages, beverage mixes and concentrates, including but not limited to, carbonated and non-carbonated beverages, alcoholic and non- alcoholic beverages, ready to drink beverages, liquid concentrate formulations for preparing beverages such as sodas, and dry powdered beverage precursor mixes. The Beverage category also includes the alcoholic drinks, the soft drinks, sports drinks, isotonic beverages, and hot drinks. The alcoholic drinks include, but are not limited to beer, cider/perry, FABs, wine, and spirits. The soft drinks include, but are not limited to carbonates, such as colas and non-cola carbonates; fruit juice, such as juice, nectars, juice drinks and fruit flavored drinks; bottled water, which includes sparkling water, spring water and purified/table water; functional drinks, which can be carbonated or still and include sport, energy or elixir drinks; concentrates, such as liquid and powder concentrates in ready to drink measure. The drinks, either hot or cold, include, but are not limited to coffee or ice coffee, such as fresh, instant, and combined coffee; tea or ice tea, such as black, green, white, oolong, and flavored tea; and other drinks including flavor-, malt- or plant-based powders, granules, blocks or tablets mixed with milk or water.

The Snack Food category generally refers to any food that can be a light informal meal including, but not limited to Sweet and savory snacks and snack bars. Examples of snack food include, but are not limited to fruit snacks, chips/crisps, extruded snacks, tortilla/corn chips, popcorn, pretzels, nuts and other sweet and savory snacks. Examples of snack bars include, but are not limited to granola/muesli bars, breakfast bars, energy bars, fruit bars and other snack bars.

The Baked Goods category generally refers to any edible product the process of preparing which involves exposure to heat or excessive sunlight. Examples of baked goods include, but are not limited to bread, buns, cookies, muffins, cereal, toaster pastries, pastries, waffles, tortillas, biscuits, pies, bagels, tarts, quiches, cake, any baked foods, and any combination thereof.

The Ice Cream category generally refers to frozen dessert containing cream and sugar and flavoring. Examples of ice cream include, but are not limited to: impulse ice cream; take-home ice cream; frozen yoghurt and artisanal ice cream; soy, oat, bean (e.g., red bean and mung bean), and rice-based ice creams.

The Confectionery category generally refers to edible product that is sweet to the taste. Examples of confectionery include, but are not limited to candies, gelatins, chocolate confectionery, sugar confectionery, gum, and the likes and any combination products.

The Meal Replacement category generally refers to any food intended to replace the normal meals, particularly for people having health or fitness concerns. Examples of meal replacement include, but are not limited to slimming products and convalescence products.

The Ready Meal category generally refers to any food that can be served as meal without extensive preparation or processing. The ready meal includes products that have had recipe “skills” added to them by the manufacturer, resulting in a high degree of readiness, completion and convenience. Examples of ready meal include, but are not limited to canned/preserved, frozen, dried, chilled ready meals; dinner mixes; frozen pizza; chilled pizza; and prepared salads.

The Pasta and Noodle category includes any pastas and/or noodles including, but not limited to canned, dried and chilled/fresh pasta; and plain, instant, chilled, frozen and snack noodles.

The Canned/Preserved Food category includes, but is not limited to canned/preserved meat and meat products, fish/seafood, vegetables, tomatoes, beans, fruit, ready meals, soup, pasta, and other canned/preserved foods.

The Frozen Processed Food category includes, but is not limited to frozen processed red meat, processed poultry, processed fish/seafood, processed vegetables, meat substitutes, processed potatoes, bakery products, desserts, ready meals, pizza, soup, noodles, and other frozen food.

The Dried Processed Food category includes, but is not limited to rice, dessert mixes, dried ready meals, dehydrated soup, instant soup, dried pasta, plain noodles, and instant noodles. The Chill Processed Food category includes, but is not limited to chilled processed meats, processed fish/seafood products, lunch kits, fresh cut fruits, ready meals, pizza, prepared salads, soup, fresh pasta and noodles.

The Sauces, Dressings and Condiments category includes, but is not limited to tomato pastes and purees, bouillon/stock cubes, herbs and spices, monosodium glutamate (MSG), table sauces, soy based sauces, pasta sauces, wet/cooking sauces, dry sauces/powder mixes, ketchup, mayonnaise, mustard, salad dressings, vinaigrettes, dips, pickled products, and other sauces, dressings and condiments.

The Baby Food category includes, but is not limited to milk- or soybean-based formula; and prepared, dried and other baby food.

The Spreads category includes, but is not limited to jams and preserves, honey, chocolate spreads, nut based spreads, and yeast based spreads.

The Dairy Product category generally refers to edible product produced from mammal's milk. Examples of dairy product include, but are not limited to drinking milk products, cheese, yoghurt and sour milk drinks, and other dairy products.

Additional examples for flavored products, particularly food and beverage products or formulations, are provided as follows. Exemplary ingestible compositions include one or more confectioneries, chocolate confectionery, tablets, countlines, bagged selflines/softlines, boxed assortments, standard boxed assortments, twist wrapped miniatures, seasonal chocolate, chocolate with toys, alfajores, other chocolate confectionery, mints, standard mints, power mints, boiled sweets, pastilles, gums, jellies and chews, toffees, caramels and nougat, medicated confectionery, lollipops, liquorice, other sugar confectionery, bread, packaged/industrial bread, unpackaged/artisanal bread, pastries, cakes, packaged/industrial cakes, unpackaged/artisanal cakes, cookies, chocolate coated biscuits, sandwich biscuits, filled biscuits, savory biscuits and crackers, bread substitutes, breakfast cereals, rte cereals, family breakfast cereals, flakes, muesli, other cereals, children's breakfast cereals, hot cereals, ice cream, impulse ice cream, single portion dairy ice cream, single portion water ice cream, multi-pack dairy ice cream, multi-pack water ice cream, take-home ice cream, take-home dairy ice cream, ice cream desserts, bulk ice cream, take-home water ice cream, frozen yoghurt, artisanal ice cream, dairy products, milk, fresh/pasteurized milk, full fat fresh/pasteurized milk, semi skimmed fresh/pasteurized milk, long-life/uht milk, full fat long life/uht milk, semi skimmed long life/uht milk, fat-free long life/uht milk, goat milk, condensed/evaporated milk, plain condensed/evaporated milk, flavored, functional and other condensed milk, flavored milk drinks, dairy only flavored milk drinks, flavored milk drinks with fruit juice, soy milk, sour milk drinks, fermented dairy drinks, coffee whiteners, powder milk, flavored powder milk drinks, cream, cheese, processed cheese, spreadable processed cheese, unspreadable processed cheese, unprocessed cheese, spreadable unprocessed cheese, hard cheese, packaged hard cheese, unpackaged hard cheese, yoghurt, plain/natural yoghurt, flavored yoghurt, fruited yoghurt, probiotic yoghurt, drinking yoghurt, regular drinking yoghurt, probiotic drinking yoghurt, chilled and shelf-stable desserts, dairy-based desserts, soy-based desserts, chilled snacks, fromage frais and quark, plain fromage frais and quark, flavored fromage frais and quark, savory fromage frais and quark, sweet and savory snacks, fruit snacks, chips/crisps, extruded snacks, tortilla/corn chips, popcorn, pretzels, nuts, other sweet and savory snacks, snack bars, granola bars, breakfast bars, energy bars, fruit bars, other snack bars, meal replacement products, slimming products, convalescence drinks, ready meals, canned ready meals, frozen ready meals, dried ready meals, chilled ready meals, dinner mixes, frozen pizza, chilled pizza, soup, canned soup, dehydrated soup, instant soup, chilled soup, hot soup, frozen soup, pasta, canned pasta, dried pasta, chilled/fresh pasta, noodles, plain noodles, instant noodles, cups/bowl instant noodles, pouch instant noodles, chilled noodles, snack noodles, canned food, canned meat and meat products, canned fish/seafood, canned vegetables, canned tomatoes, canned beans, canned fruit, canned ready meals, canned soup, canned pasta, other canned foods, frozen food, frozen processed red meat, frozen processed poultry, frozen processed fish/seafood, frozen processed vegetables, frozen meat substitutes, frozen potatoes, oven baked potato chips, other oven baked potato products, non-oven frozen potatoes, frozen bakery products, frozen desserts, frozen ready meals, frozen pizza, frozen soup, frozen noodles, other frozen food, dried food, dessert mixes, dried ready meals, dehydrated soup, instant soup, dried pasta, plain noodles, instant noodles, cups/bowl instant noodles, pouch instant noodles, chilled food, chilled processed meats, chilled fish/seafood products, chilled processed fish, chilled coated fish, chilled smoked fish, chilled lunch kit, chilled ready meals, chilled pizza, chilled soup, chilled/fresh pasta, chilled noodles, oils and fats, olive oil, vegetable and seed oil, cooking fats, butter, margarine, spreadable oils and fats, functional spreadable oils and fats, sauces, dressings and condiments, tomato pastes and purees, bouillon/stock cubes, stock cubes, gravy granules, liquid stocks and fonds, herbs and spices, fermented sauces, soy based sauces, pasta sauces, wet sauces, dry sauces/powder mixes, ketchup, mayonnaise, regular mayonnaise, mustard, salad dressings, regular salad dressings, low fat salad dressings, vinaigrettes, dips, pickled products, other sauces, dressings and condiments, baby food, milk formula, standard milk formula, follow-on milk formula, toddler milk formula, hypoallergenic milk formula, prepared baby food, dried baby food, other baby food, spreads, jams and preserves, honey, chocolate spreads, nut-based spreads, and yeast-based spreads. Exemplary ingestible compositions also include confectioneries, bakery products, ice creams, dairy products, sweet and savory snacks, snack bars, meal replacement products, ready meals, soups, pastas, noodles, canned foods, frozen foods, dried foods, chilled foods, oils and fats, baby foods, or spreads or a mixture thereof. Exemplary ingestible compositions also include breakfast cereals, sweet beverages or solid or liquid concentrate compositions for preparing beverages, ideally so as to enable the reduction in concentration of previously known saccharide sweeteners, or artificial sweeteners.

Some embodiments provide a chewable composition that may or may not be intended to be swallowed. In some embodiments, the chewable composition may be gum, chewing gum, sugarized gum, sugar-free gum, functional gum, bubble gum including compounds as disclosed and described herein, individually or in combination.

Typically at least a sweet receptor modulating amount, a sweet receptor ligand modulating amount, a sweet flavor modulating amount, a sweet flavoring agent amount, a sweet flavor enhancing amount, or a therapeutically effective amount of one or more of the present compounds will be added to the ingestible composition, optionally in the presence of sweeteners so that the sweet flavor modified ingestible composition has an increased sweet taste as compared to the ingestible composition prepared without the compounds of the present invention, as judged by human beings or animals in general, or in the case of formulations testing, as judged by a majority of a panel of at least eight human taste testers, via procedures commonly known in the field.

In some embodiments, compounds as disclosed and described herein, individually or in combination, modulate the sweet taste or other taste properties of other natural or synthetic sweet tastants, and ingestible compositions made therefrom. In one embodiment, the compounds as disclosed and described herein, individually or in combination, may be used or provided in its ligand enhancing concentration(s). For example, the compounds as disclosed and described herein, individually or in combination, may be present in an amount of from 0.001 ppm to 100 ppm, or narrower alternative ranges from 0.1 ppm to 50 ppm, from 0.01 ppm to 40 ppm, from 0.05 ppm to 30 ppm, from 0.01 ppm to 25 ppm, or from 0.1 ppm to 30 ppm, or from 0.1 ppm to 25 ppm, or from 1 ppm to 30 ppm, or from 1 ppm to 25 ppm.

In some embodiments, flavor-modifying compounds as disclosed and described herein, individually or in combination, may be provided in a flavoring concentrate formulation, e.g., suitable for subsequent processing to produce a ready-to-use (i.e., ready-to-serve) product. By “a flavoring concentrate formulation”, it is meant a formulation which should be reconstituted with one or more diluting medium to become a ready-to-use composition. The term “ready-to-use composition” is used herein interchangeably with “ingestible composition”, which denotes any substance that, either alone or together with another substance, can be taken by mouth whether intended for consumption or not. In one embodiment, the ready-to-use composition includes a composition that can be directly consumed by a human or animal. The flavoring concentrate formulation is typically used by mixing with or diluted by one or more diluting medium, e.g., any consumable or ingestible ingredient or product, to impart or modify one or more flavors to the diluting medium. Such a use process is often referred to as reconstitution. The reconstitution can be conducted in a household setting or an industrial setting. For example, a frozen fruit juice concentrate can be reconstituted with water or other aqueous medium by a consumer in a kitchen to obtain the ready-to-use fruit juice beverage. In another example, a soft drink syrup concentrate can be reconstituted with water or other aqueous medium by a manufacturer in large industrial scales to produce the ready-to-use soft drinks. Since the flavoring concentrate formulation has the flavoring agent or flavor modifying agent in a concentration higher than the ready-to-use composition, the flavoring concentrate formulation is typically not suitable for being consumed directly without reconstitution. There are many benefits of using and producing a flavoring concentrate formulation. For example, one benefit is the reduction in weight and volume for transportation as the flavoring concentrate formulation can be reconstituted at the time of usage by the addition of suitable solvent, solid or liquid.

The flavored products set forth according to any of the foregoing embodiments, also include, in certain embodiments, one or more additional flavor-modifying compounds, such as compounds that enhance sweetness (e.g., hesperetin, naringenin, glucosylated steviol glycosides, etc.), compounds that block bitterness, compounds that enhance umami, compounds that reduce sourness, compounds that enhance saltiness, compounds that enhance a cooling effect, or any combinations of the foregoing.

In certain embodiments of any aspects and embodiments set forth herein that refer to a sweetening or flavoring concentrate, the sweetening or flavoring concentrate is a non-naturally-occurring product, such as a composition specifically manufactured for the production of a flavored product, such as food or beverage product.

In one embodiment, the flavoring concentrate formulation comprises i) compounds as disclosed and described herein, individually or in combination; ii) a carrier; and iii) optionally at least one adjuvant. The term “carrier” denotes a usually inactive accessory substance, such as solvents, binders, or other inert medium, which is used in combination with the present compound and one or more optional adjuvants to form the formulation. For example, water or starch can be a carrier for a flavoring concentrate formulation. In some embodiments, the carrier is the same as the diluting medium for reconstituting the flavoring concentrate formulation; and in other embodiments, the carrier is different from the diluting medium. The term “carrier” as used herein includes, but is not limited to, ingestibly acceptable carrier.

The term “adjuvant” denotes an additive which supplements, stabilizes, maintains, or enhances the intended function or effectiveness of the active ingredient, such as the compound of the present invention. In one embodiment, the at least one adjuvant comprises one or more flavoring agents. The flavoring agent may be of any flavor known to one skilled in the art or consumers, such as the flavor of chocolate, coffee, tea, mocha, French vanilla, peanut butter, chai, or combinations thereof. In another embodiment, the at least one adjuvant comprises one or more sweeteners. The one or more sweeteners can be any of the sweeteners described in this application. In another embodiment, the at least one adjuvant comprises one or more ingredients selected from the group consisting of a emulsifier, a stabilizer, an antimicrobial preservative, an antioxidant, vitamins, minerals, fats, starches, protein concentrates and isolates, salts, and combinations thereof. Examples of emulsifiers, stabilizers, antimicrobial preservatives, antioxidants, vitamins, minerals, fats, starches, protein concentrates and isolates, and salts are described in U.S. Pat. No. 6,468,576, the content of which is hereby incorporated by reference in its entirety for all purposes.

In one embodiment, the present flavoring concentrate formulation can be in a form selected from the group consisting of liquid including solution and suspension, solid, foamy material, paste, gel, cream, and a combination thereof, such as a liquid containing certain amount of solid contents. In one embodiment, the flavoring concentrate formulation is in form of a liquid including aqueous-based and nonaqueous-based. In some embodiments, the present flavoring concentrate formulation can be carbonated or non-carbonated.

The flavoring concentrate formulation may further comprise a freezing point depressant, nucleating agent, or both as the at least one adjuvant. The freezing point depressant is an ingestibly acceptable compound or agent which can depress the freezing point of a liquid or solvent to which the compound or agent is added. That is, a liquid or solution containing the freezing point depressant has a lower freezing point than the liquid or solvent without the freezing point depressant. In addition to depress the onset freezing point, the freezing point depressant may also lower the water activity of the flavoring concentrate formulation. The examples of the freezing point depressant include, but are not limited to, carbohydrates, oils, ethyl alcohol, polyol, e.g., glycerol, and combinations thereof. The nucleating agent denotes an ingestibly acceptable compound or agent which is able to facilitate nucleation. The presence of nucleating agent in the flavoring concentrate formulation can improve the mouthfeel of the frozen Blushes of a frozen slush and to help maintain the physical properties and performance of the slush at freezing temperatures by increasing the number of desirable ice crystallization centers. Examples of nucleating agents include, but are not limited to, calcium silicate, calcium carbonate, titanium dioxide, and combinations thereof.

In one embodiment, the flavoring concentrate formulation is formulated to have a low water activity for extended shelf life. Water activity is the ratio of the vapor pressure of water in a formulation to the vapor pressure of pure water at the same temperature. In one embodiment, the flavoring concentrate formulation has a water activity of less than about 0.85. In another embodiment, the flavoring concentrate formulation has a water activity of less than about 0.80. In another embodiment, the flavoring concentrate formulation has a water activity of less than about 0.75.

In one embodiment, the flavoring concentrate formulation has the present compound in a concentration that is at least 2 times of the concentration of the compound in a ready-to-use composition. In one embodiment, the flavoring concentrate formulation has the present compound in a concentration that is at least 5 times of the concentration of the compound in a ready-to-use composition. In one embodiment, the flavoring concentrate formulation has the present compound in a concentration that is at least 10 times of the concentration of the compound in a ready-to-use composition. In one embodiment, the flavoring concentrate formulation has the present compound in a concentration that is at least 15 times of the concentration of the compound in a ready-to-use composition. In one embodiment, the flavoring concentrate formulation has the present compound in a concentration that is at least 20 times of the concentration of the compound in a ready-to-use composition. In one embodiment, the flavoring concentrate formulation has the present compound in a concentration that is at least 30 times of the concentration of the compound in a ready-to-use composition. In one embodiment, the flavoring concentrate formulation has the present compound in a concentration that is at least 40 times of the concentration of the compound in a ready-to-use composition. In one embodiment, the flavoring concentrate formulation has the present compound in a concentration that is at least 50 times of the concentration of the compound in a ready-to-use composition. In one embodiment, the flavoring concentrate formulation has the present compound in a concentration that is at least 60 times of the concentration of the compound in a ready-to-use composition. In one embodiment, the flavoring concentrate formulation has the present compound in a concentration that is up to 100 times of the concentration of the compound in a ready-to-use composition.

The sweetening or flavoring concentrates set forth according to any of the foregoing embodiments, also include, in certain embodiments, one or more additional flavor-modifying compounds, such as compounds that enhance sweetness (e.g., hesperetin, naringenin, glucosylated steviol glycosides, etc.), compounds that block bitterness (e.g., eriodictyol, homoeriodictyol, sterubin, and salts or glycoside derivatives thereof, as well as vanillyl lignans, e.g., matairesinol and other compounds set forth in PCT Publication No. WO 2012/146584), compounds that enhance umami (e.g., rubemamine, rubescenamine, (E)-3-(3,4-dimethoxyphenyl)-N-(4-methoxyphenethyl)acrylamide, and the like), compounds that reduce sourness and/or licorice taste, compounds that enhance saltiness, compounds that enhance a cooling effect, or any combinations of the foregoing.

### Methods of Preparation

The compounds disclosed herein may be synthesized by methods described below, or by modification of these methods. Ways of modifying the methodology include, among others, temperature, solvent, reagents etc., known to those skilled in the art. In general, during any of the processes for preparation of the compounds disclosed herein, it may be necessary or desirable to protect sensitive or reactive groups on any of the molecules concerned.

In certain aspects, the disclosure provides methods of making flavor-modifying compounds of the foregoing aspects via an enzyme-catalyzed process. For example, in certain embodiments, the methods include providing a precursor, such as glycyrrhizin, or a derivative thereof. In some embodiments, the glycyrrhizin is synthesized through enzymatic processes characteristic of those for enzymatically catatyzed syntheses of terpene compounds. The glycyrrhizin can be further derivatized in any suitable manner For example, in some embodiments, the monoglucuronate derivative of glycyrrhizin is synthesized by catalytically hydrolyzing the ether linkage between the two glucuronyl moieties to remove the distal of the two moieties. Further derivatives can be made, such as adding an —OH moiety at the positions identified by R¹ and R² in the compound of formula (I) or formula (Ia), by conventional synthetic means.

### EXAMPLES

To further illustrate this invention, the following examples are included. The examples should not, of course, be construed as specifically limiting the invention. Variations of these examples within the scope of the claims are within the purview of one skilled in the art and are considered to fall within the scope of the invention as described, and claimed herein. The reader will recognize that the skilled artisan, armed with the present disclosure, and skill in the art is able to prepare and use the invention without exhaustive examples.

### Example 1

### Sweetness Enhancement

A test compounds were tested for its sweetness enhancement of a 5% (w/w) aqueous solution of sucrose. Taste panelists were asked to taste the following 5% sucrose solutions in Henniez water and rate their sweetness on a scale from 0 to 10, and to rate the presence of any licorice taste in the sample on a scale from 0 to 10. Results are set forth in Table 2.

TABLE 2

| Compound (conc) | Sweetness (0-10) | Licorice Taste (0-10) |
|---|---|---|
| No flavor modifier | 5.0 | 0.0 |
| 108 (50 ppm) | 5.8 | 0.5 |
| 109 + 110 (4:1) (100 ppm) | 8.0 | 2.0 |

### Example 2

### Compound 108 Preparation by Biotransformation

The description below sets forth a non-limiting method by which the compound 101 was obtained.

β-Glycyrrhizin (Fluka, 1 g) was diluted in 50 mL of water under gentle heating to prevent gelation. Then, 1 mL of Pectinex Ultra SP-L (Novozymes) was added and the reaction was stirred for 2 days at 40° C. in an orbital shaker. The conversion rate was 30%. Then, 20 mL of water was added to compensate for the evaporation and the reaction was stirred for an additional 7 days. At that point, the conversion rate was 45%, and not much glycyrrhetinic acid was formed. The reaction was stopped by inactivating the enzyme at 95° C. for 10 minutes. Water was added (500 mL) and the reaction was filtered over a glass fiber disc and freeze-dried at yield about 1 g (100%) of white powder. Compound 108 was purified by preparative HPLC in about 100 consecutive injections over a C18 column eluted using 60% acetonitrile (0.1% formic acid). Alternatively, it could also be prepared by flash chromatography over RP-18 silicagel eluted with a gradient of water and acetonitrile (from 35% to 55% ACN). ¹³C NMR (150 MHz, Pyr): δ 16.8 (q), 17.0 (q), 17.6 (t), 18.8 (q), 23.6 (q), 26.6 (t), 26.8 (t), 26.9 (t), 28.2 (q), 28.7 (q), 28.8 (q), 31.6 (t), 32.2 (s), 32.9 (t), 37.3 (s), 38.5 (t), 39.5 (t), 40.0 (s), 41.7 (t), 43.5 (s), 44.1 (s), 45.6 (s), 48.7 (d), 55.3 (d), 62.1 (d), 73.5 (d), 75.6 (d), 77.9 (d), 78.2 (d), 88.8 (d), 107.3 (d), 128.7 (d), 169.6 (s), 172.8 (s), 179.2 (s), 199.5 (s).

### Example 3

### Compounds 109/110 Preparation by Biotransformation and Chemical Synthesis

The description below sets forth a non-limiting method by which the Compounds 109 and 110 were obtained.

The same protocol as for the preparation of 108 was followed except the mixture of 109/110 was obtained from a flash chromatography over RP-18 silicagel eluted with a gradient of water and acetonitrile (35 to 55% ACN). The mixture contained 80% of 109 and 20% of 110. ¹³C NMR (150 MHz, Pyr): δ 16.4 (q), 18.1 (t), 18.6 (q), 23.4 (q), 23.5 (q), 26.6 (t), 26.8 (t), 26.9 (t), 28.7 (q), 28.8 (q), 31.6 (t), 32.2 (s), 33.2 (t), 37.0 (s), 38.4 (t), 39.3 (t), 41.7 (t), 43.5 (s), 44.1 (s), 44.8 (s), 45.5 (s), 48.7 (d), 55.8 (d), 61.9 (d), 63.3 (t), 73.5 (d), 75.4 (d), 78.2 (d), 88.8 (d), 106.5 (d), 128.7 (d), 169.7 (s), 172.6 (s), 179.2 (s), 199.4 (s).

1. A method of enhancing a sweet taste of an ingestible composition, the method comprising introducing a flavor-modifying compound to the ingestible composition at a concentration ranging from 5 ppm to 200 ppm, based on the total weight of the ingestible composition, wherein the flavor-modifying compound is a compound of formula (I): 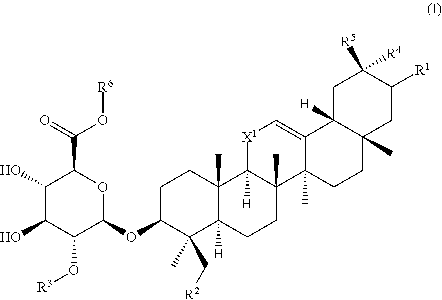
or a salt thereof, wherein:
R¹ is a hydrogen atom, —OH, or —O—R^{1A};
R^{1A} is C₁₋alkyl, which is optionally substituted one or more times by substituents selected from the group consisting of —OH and C₁₋₆alkoxy;
R² is a hydrogen atom, —OH, or —O—R^{2A};
C₁₋₆ alkyl, which is optionally substituted one or more times by substituents selected from the group consisting of —OH and C₁₋₆alkoxy; or R^{2A} is —C(O)—(C₁₋₆ alkyl) or a glucosyl moiety;
R³ is a hydrogen atom, a glucuronyl moiety, a C₁₋₆ glucuronyl ester moiety, or a glucosyl moiety;
one of R⁴ and R⁵ is a methyl group, and the other is —C(O)—O—R^{4A};
R⁴A is a hydrogen atom, a glucuronyl moiety, a C₁₋₆ glucuronyl ester moiety, or a glucosyl moiety;
R⁶ is a hydrogen atom or C₁₋₆ alkyl; and
X¹ is —CH₂— or >C═O.

2. The method of claim 1, wherein R⁴ is a methyl group and R⁵ is —C(O)—O—R^{4A}.

3. The method of claim 2, wherein R^{4A} is a hydrogen atom.

4. The method of claim 1, wherein X¹ is >C═O.

5. The method of any one of claims 1 to 4, wherein R³ is a hydrogen atom.

6. The method of any one of claims 1 to 5, wherein R¹ is a hydrogen atom.

7. The method of any one of claims 1 to 5, wherein R¹ is —OH.

8. The method of any one of claims 1 to 7, wherein R² is a hydrogen atom.

9. The method of any one of claims 1 to 7, wherein R² is —OH.

10. The method of claim 1, wherein the flavor-modifying compound is a compound of formula (Ia): 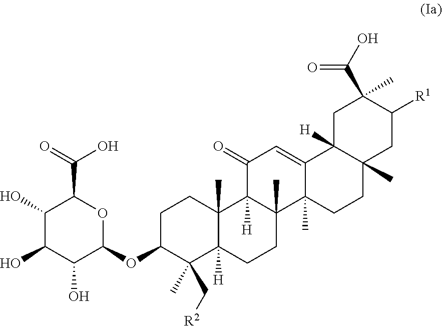
or a salt thereof.

11. (canceled)

12. The method of any one of claims 1 to 11, wherein the use is to enhance the sweet taste of an ingestible composition.

13. The method of any one of claims 1 to 12, wherein the ingestible composition comprises a sweetener.

14. (canceled)

15. An ingestible composition, which is a non-naturally occurring composition and comprises a flavor-modifying compound at a concentration ranging from 5 ppm to 200 ppm, based on the total weight of the ingestible composition, and wherein the flavor-modifying compound is a compound of formula (I): 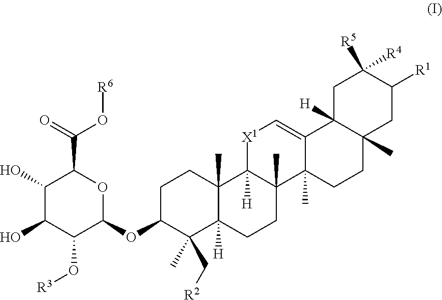
or a salt thereof, wherein:
R¹ is a hydrogen atom, —OH, or —O—R^{1A};
R^{1A} is C₁-₆ alkyl, which is optionally substituted one or more times by substituents selected from the group consisting of —OH and C₁₋₆alkoxy;
R² is a hydrogen atom, —OH, or —O—R^{2A};
R^{2A} is C₁₋₆ alkyl, which is optionally substituted one or more times by substituents selected from the group consisting of —OH and C₁₋₆alkoxy; or R^{2A} is —C(O)—(C₁₋₆alkyl) or a glucosyl moiety;
R³ is a hydrogen atom, a glucuronyl moiety, a C₁₋₆glucuronyl ester moiety, or a glucosyl moiety;
one of R⁴ and R⁵ is a methyl group, and the other is —C(O)—O—R^{4A};
R^{4A} is a hydrogen atom, a glucuronyl moiety, a C₁₋₆glucuronyl ester moiety, or a glucosyl moiety;
R⁶ is a hydrogen atom or C₁₋₆ alkyl; and
X¹ is —CH₂— or >C═O.

16. The composition of claim 15, further comprising one or more sweeteners selected from the group consisting of sucrose, fructose, glucose, erythritol, xylitol, and any combinations thereof.

17. The composition of claim 15, wherein the flavor-modifying compound is a compound of formula (Ia): 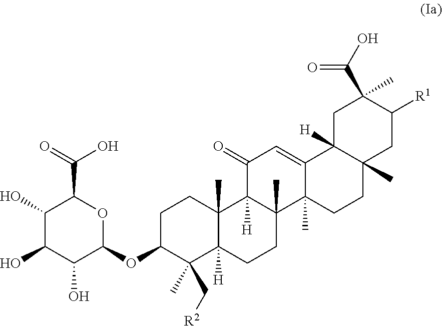
or salt; a salt thereof.
